(19) United States
(12) Patent Application Publication
(10) Pub. No.: US 2026/0027355 A1
(43) Pub. Date: Jan. 29, 2026

(54) SYSTEM AND METHOD FOR CHARACTERIZATION OF AN ORGANIC MEDIUM SURROUNDING AN ELECTRODE

(71) Applicant: BIOTRONIK SE & Co. KG, Berlin (DE)

(72) Inventors: Joel Alain TSEMO KAMGA, Berlin (DE); Gernot KOLBERG, Berlin (DE)

(73) Assignee: BIOTRONIK SE & Co. KG, Berlin (DE)

(21) Appl. No.: 18/998,564

(22) PCT Filed: Jul. 14, 2023
(86) PCT No.: PCT/EP2023/069651

§ 371 Date: Jan. 27, 2025

(30) Foreign Application Priority Data
Aug. 9, 2022 (EP) 22189361.3

(51) Int. Cl. A61N 1/08; A61N 1/05

(52) U.S. Cl. CPC A61N 1/08; A61N 1/059; A61N 2001/083

(57) ABSTRACT

A system for characterization of an organic medium surrounding an electrode, comprising: two or more electrodes, wherein at least one electrode of the two or more electrodes is configured to be insertable in tissue; and an analysis unit connectable to the two or more electrodes and configured to apply a measurement signal to the two or more electrodes, wherein the analysis unit is further configured to determine an impedance between the two or more electrodes based on the measurement signal and characterize the organic medium surrounding the at least one electrode based on an amplitude and/or phase of the impedance or real part and/or imaginary part of the impedance.

### CROSS-REFERENCE TO RELATED APPLICATIONS

This application is the United States National Phase under 35 U.S.C. § 371 of PCT International Patent Application No. PCT/EP2023/069651, filed on Jul. 14, 2023, which claims the benefit of European Patent Application No. 22189361.3, filed on Aug. 9, 2022, the disclosures of which are hereby incorporated by reference herein in their entireties.

### TECHNICAL FIELD

Embodiments of the present disclosure relate to a system for characterization of an organic medium surrounding an electrode, a method for characterization of an organic medium surrounding an electrode, and a machine-readable medium for executing the method. Embodiments of the present disclosure relate more particularly to an electrical characterization of a penetration depth of an electrode and/or a diagnostic tool in tissue.

### BACKGROUND

There are many known medical devices that can be permanently or temporarily inserted into a body. Examples of such medical devices include medical implants, diagnostic tools and combinations thereof. Medical implants are widely used to replace, support and/or enhance biological structures of patients. Examples of medical implants include, but are not limited to, cardiovascular medical devices such as artificial hearts, artificial heart valves, implantable cardioverter-defibrillators, cardiac pacemakers, implantable sensors, and coronary stents.

Some medical devices include, or are connected to, electrodes that must be inserted into organic tissue. For example, a cardiac pacemaker implanted in a subcutaneous pocket can be connected to pacing leads which carry electrodes. The electrodes of the pacing leads can be placed in the heart to help the heart beat more evenly.

When implanting the electrodes, physicians are assisted by various means or procedures that allow visualization of the electrodes inside the heart. These means include X-ray or mapping (sensing, stimulus threshold). The penetration depth of the fixation in the myocardium cannot be assessed, which may result in an increase in the stimulus threshold as well as possible dislocation of the electrodes. Especially if the electrode tip for deep septal stimulation is inserted deeply into the tissue, there are no suitable indicators other than sensing and stimulus threshold that tell the physician whether the tip is fully inserted, how deep it is, or whether it comes out again on the other side in the left ventricle.

U.S. Pat. No. 11,045,653 B1 describes improved pacing leads, adapters for connecting to a conventional pacemaker and methods for left bundle branch (LBB) pacing with closely monitored depth control during electrode implantation. Impedance measurements may be obtained using various measurement parameters. Frequency of test impulses is one of these parameters characterizing the impedance measurement process.

European Patent No. 3 435 858 B1 relates to devices to assess infarcted tissue by measuring electrical impedance. More specifically, the devices may be used to recognize the extent and deepness of an infarcted tissue, such as, for example, myocardial infarcted tissue. The aim, to provide a measuring device for medical applications, which can be used to characterize tissues, is achieved by measuring the changes in impedance during the entire cardiac cycle by injecting electrical current with a broadband spectrum.

In light of the above, a system for characterization of an organic medium surrounding an electrode, a method for characterization of an organic medium surrounding an electrode, and a machine-readable medium for executing the method that overcome at least some of the problems in the art are beneficial.

The present disclosure is directed toward overcoming one or more of the above-mentioned problems, though not necessarily limited to embodiments that do.

### SUMMARY

It is an object of the present disclosure to provide a system for characterization of an organic medium surrounding an electrode, a method for characterization of an organic medium surrounding an electrode, and a machine-readable medium for executing the method that allow a precise placement of electrode(s) in organic tissue. Another object of the present disclosure is to prevent dislocation of the electrode(s) and/or to reduce power consumption of medical devices and/or to prevent patient harm, such as puncture of the myocardium.

At least the objects are solved by the features of the independent claims. Preferred embodiments are defined in the dependent claims.

According to an independent aspect of the present disclosure, a system for characterization of an organic medium surrounding an electrode is provided. The system includes two or more electrodes, wherein at least one (first) electrode of the two or more electrodes is configured to be inserted in tissue; and an analysis unit connectable to the two or more electrodes and configured to apply a measurement signal to the two or more electrodes, wherein the analysis unit is further configured to determine an impedance between the two or more electrodes based on the measurement signal and characterize the organic medium surrounding the at least one electrode based on an amplitude and/or phase of the impedance and/or wherein the analysis unit is configured to apply a current pulse to at least one (first) electrode and wherein the analysis unit is further configured to characterize the organic medium surrounding the at least one electrode based on the morphology of the applied current pulse.

Preferably, the organic medium is selected from the group including (or consisting of) tissue and blood. The tissue may be cardiac tissue, such as a myocardial wall.

According to some embodiments, which can be combined with other embodiments described herein, the measurement signal can be a dedicated or separate signal for determining the impedance. In other embodiments, the measurement signal can be a temporal signal, such as a pacemaker pulse.

According to some embodiments, which can be combined with other embodiments described herein, the two or more electrodes include at least one first electrode and at least one second electrode.

Preferably, the at least one first electrode is configured to be inserted in the tissue.

Additionally, or alternatively, the at least one second electrode is spaced apart from the first electrode.

According to some embodiments, which can be combined with other embodiments described herein, the at least one first electrode is selected from the group including (or consisting of) a screw electrode, a tip electrode, and a hook electrode.

Preferably, the screw electrode is configured to engage with the tissue upon rotation.

Preferably, the tip electrode has a pointed tip insertable into the tissue. Additionally, or alternatively, the tip electrode is a passively attachable electrode.

Preferably, the hook electrode has a bent portion insertable into the tissue.

According to some embodiments, which can be combined with other embodiments described herein, the at least one second electrode is selected from the group including (or consisting of) a ring electrode, a housing, and an external electrode attachable to a body.

Preferably, the ring electrode is an electrode of a screw electrode (the at least one first electrode) and/or an electrode having a fixed potential and/or an electrically active electrode.

Preferably, the housing is a housing of a power unit or a housing of the analysis unit.

Preferably, the external electrode is temporarily attachable to a patient's body, for example, the patient's skin.

According to some embodiments, which can be combined with other embodiments described herein, the analysis unit is configured to sample and/or measure and/or determine the impedance over a predetermined frequency range (or frequency band).

Preferably, the predetermined frequency range (or frequency band) is from 1 kHz (or 50 Hz) to 10 MHz, preferably from 1 kHz (or 50 Hz) to 1 MHz. Additionally, or alternatively, the predetermined frequency range can have a width of (or extend over) 300 kHz or more, 500 kHz or more, 800 kHz or more, or 1 MHz or more.

According to some embodiments, which can be combined with other embodiments described herein, the analysis unit is configured to sample and/or measure and/or determine the impedance at at least one frequency point (e.g., a frequency point characteristic for the phase at which the first electrode is completely in the tissue), this frequency point being in a frequency range between 100 kHz and 400 kHz, preferably between 200 kHz and 350 kHz, and particularly preferably between 250 kHz and 300 kHz. Optimally, the frequency point is at 275 kHz. In the following “frequency point” is also called “certain frequency”.

It is noted that the present disclosure is not limited thereto and that, instead of sampling over a predetermined frequency range, a pulse or step response can be used in the analysis. For example, a current response to a rectangular or otherwise shaped voltage pulse can be evaluated, or a voltage response for a controlled current pulse can be evaluated. A Fourier analysis would then give the connection to the frequencies to allow the analysis described in the following.

According to some embodiments, which can be combined with other embodiments described herein, the analysis unit is configured to determine a number of local minima (relaxations) in the phase (i.e., the phase curve) or in the imaginary part of the impedance over the predetermined frequency range, and to determine an organic medium surrounding the at least one electrode based on the number of local minima.

Preferably, the analysis unit is configured to determine that the at least one electrode is located in a first organic medium if the number of local minima is n, and to determine that the at least one electrode is located at least partially in a second organic medium if the number of local minima is m, wherein m≠n. For example, intermediate states can be detected, e.g., whether an electrode is completely in the tissue or still partially in the blood.

The second organic medium is different from the first organic medium. For example, the first organic medium is blood, and the second organic medium is tissue such as a myocardial wall. In this example, m n.

According to some embodiments, which can be combined with other embodiments described herein, the analysis unit is configured to determine a maximum value of the amplitude of the impedance in the predetermined frequency range, and to determine an organic medium surrounding the at least one electrode based on the maximum value. For example, the analysis unit can be configured to distinguish between blood and tissue based on the maximum value.

According to some embodiments, which can be combined with other embodiments described herein, the analysis unit is configured to measure and/or determine the impedance at a reference frequency.

Preferably, the analysis unit is configured to determine an organic medium surrounding the at least one electrode based on the amplitude and/or phase of the impedance at the reference frequency.

Preferably, the analysis unit is configured to determine an organic medium surrounding the at least one electrode based on an imaginary part and/or real part of the impedance at the reference frequency.

Preferably, the reference frequency is in a range from 1 kHz (or 50 Hz) to 10 MHZ, preferably from 1 kHz (or 50 Hz) to 1 MHz.

Preferably, the reference frequency corresponds to a relaxation frequency of a cardiac muscle, such as a myocardial wall.

According to some embodiments, which can be combined with other embodiments described herein, the system includes a pacing lead carrying the two or more electrodes. The pacing lead may be a bipolar pacing lead.

Preferably, the system includes a pacemaker connected to the pacing lead. In some embodiments, the analysis unit can be included in the pacemaker. In other embodiments, the analysis unit can be provided remote from the pacemaker. Preferably, the analysis/measurement is performed at implantation, before the pacemaker (or defibrillator) is connected.

Preferably, complete impedance and/or phase curves are evaluated in some embodiments of the present disclosure. However, the present disclosure is not limited thereto and pulse shapes rather that complete impedance and/or phase curves can be evaluated in other embodiments of the present disclosure.

According to another independent aspect of the present disclosure, a method for characterization of an organic medium surrounding an electrode is provided. The method includes applying a measurement signal to two or more electrodes, wherein at least one electrode of the two or more electrodes is configured to be insertable in tissue; determining an impedance between the two or more electrodes based on the measurement signal; and characterizing the organic medium surrounding the at least one electrode based on an amplitude and/or phase of the impedance.

According to another independent aspect of the present disclosure, a machine-readable medium is provided. The machine-readable medium includes instructions executable by one or more processors to implement the method for characterization of an organic medium surrounding an electrode of the embodiments of the present disclosure.

The (e.g., non-transitory) machine readable medium may include, for example, optical media such as CD-ROMs and digital video disks (DVDs), and semiconductor memory devices such as Electrically Programmable Read-Only Memory (EPROM), and Electrically Erasable Programmable Read-Only Memory (EEPROM). The machine-readable medium may be used to tangibly retain computer program instructions or code organized into one or more modules and written in any desired computer programming language. When executed by, for example, one or more processors such computer program code may implement one or more of the methods described herein.

Embodiments are also directed at systems/devices for carrying out the disclosed methods and include system/device aspects for performing each described method aspect. These method aspects may be performed by way of hardware components, a computer programmed by appropriate software, by any combination of the two or in any other manner. Furthermore, embodiments according to the present invention are also directed at methods for operating the described device/system. It includes method aspects for carrying out every function of the device/system.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

So that the manner in which the above recited features of the present disclosure can be understood in detail, a more particular description of the disclosure, briefly summarized above, may be had by reference to embodiments. The accompanying drawings relate to embodiments of the disclosure and are described in the following:

FIG. 1 shows a schematic view of an electrode-equipped pacing lead according to embodiments of the present disclosure;

FIG. 2A-2D show different situations which may occur during implantation of the electrode-equipped pacing lead;

FIG. 3 shows a system for characterization of an organic medium surrounding an electrode according to embodiments of the present disclosure;

FIG. 4 shows a macroscopically simplified equivalent circuit of an active lead according to embodiments of the present disclosure;

FIG. 5A shows a macroscopically simplified equivalent circuit of a lead whose active electrode is located in the blood;

FIG. 5B shows a phase over a frequency range based on the equivalent circuit of FIG. 5A;

FIG. 6A shows a macroscopically simplified equivalent circuit of a lead whose active electrode is located in the tissue;

FIG. 6B shows a phase over a frequency range based on the equivalent circuit of FIG. 6A:

FIG. 7 shows maximum values of the amplitude for the cases “BloodIn”, “TouchIn”, “BloodOut”, “Half”, and “Full”:

FIG. 8A shows an amplitude of an impedance over a frequency range obtained in a real experiment;

FIG. 8B shows a phase of an impedance over a frequency range obtained in a real experiment:

FIG. 9A shows a normalized real part and a normalized imaginary part of an impedance at 300 kHz (relaxation frequency of the cardiac muscle from FIG. 8B) obtained in a real experiment. All values are normalized to the value of the case in FIG. 1:

FIG. 9B shows the mean value±standard deviation of a normalized imaginary part of FIG. 9A for different cases:

FIG. 9C shows the mean value±standard deviation of the imaginary part of FIG. 9A which has not been normalized; and

FIG. 10 shows a process of fixing an electrode in tissue according to an embodiment of the present disclosure.

### DETAILED DESCRIPTION

Reference will now be made in detail to the various embodiments of the disclosure, one or more examples of which are illustrated in the figures. Within the following description of the drawings, the same reference numbers refer to same components. Generally, only the differences with respect to individual embodiments are described. Each example is provided by way of explanation of the disclosure and is not meant as a limitation of the disclosure. Further, features illustrated or described as part of one embodiment can be used on or in conjunction with other embodiments to yield yet a further embodiment. It is intended that the description includes such modifications and variations.

The following description of preferred embodiments relates to an electrode-equipped pacing lead that can be connected to a cardiac pacemaker. However, the present disclosure is not limited thereto, and other medical devices having (or being connected to) electrodes for implantation in tissue may benefit from the embodiments described herein.

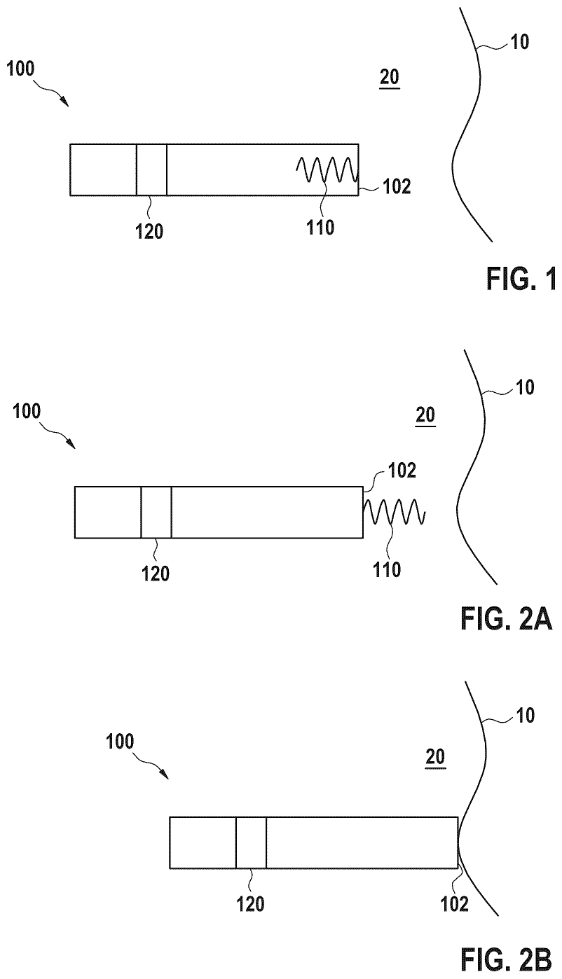

FIG. 1 shows an electrode-equipped pacing lead 100 according to embodiments of the present disclosure.

The electrode-equipped pacing lead 100 may be a bipolar lead which can be deployed into the interventricular septum from the right ventricle. In particular, the electrode-equipped pacing lead 100 may have a two-electrode connector configured for secure attachment to a cardiac pacemaker.

The pacing lead 100 includes two or more electrodes, wherein at least one electrode of the two or more electrodes is configured to be insertable in tissue, such as a myocardial wall 10. In the example of FIG. 1, blood 20 surrounds the pacing lead 100.

The two or more electrodes may include at least one first electrode 110 and at least one second electrode 120. The at least one first electrode 110 may be configured to engage with the cardiac tissue of the myocardial wall 10. For example, the at least one first electrode 110 may be arranged at a distal end 102 of the pacing lead 100. The at least one second electrode 120 can be spaced apart from the at least one first electrode 110 by a predetermined distance and does not engage with the cardiac tissue of the myocardial wall 10.

In some embodiments, the at least one first electrode 110 is selected from the group including a screw electrode, a tip electrode, and a hook electrode. In the example of FIG. 1, the at least one first electrode 110 is an active helix of a screw electrode configured to engage with the cardiac tissue upon rotation. In FIG. 1, the screw electrode is shown in a retracted or non-deployed configuration.

The at least one second electrode 120 may be selected from the group including a ring electrode, a housing, and an external electrode attachable to a patient's body. In the example of FIG. 1, the at least one second electrode 120 is an electrode of the screw electrode 110.

The embodiments of the present discourse allow for a characterization of an organic medium surrounding the at least one first electrode 110 by analyzing changes of a complex impedance signal (amplitude and/or phase) derived from a measurement signal applied to the at least one first electrode 110 and the at least one second electrode 120. Thereby, an indicator is provided which can be used to differentiate information on the relative position of the electrode(s). In particular, it can be detected whether, for example, the screw electrode is completely inside the myocardial wall 10 or (partially) still in the blood 20.

For the above characterization process, a plurality of different situations can be considered which may occur during implantation of the electrode-equipped pacing lead 100. FIG. 1 shows a situation in which the at least one first electrode 110 is in a non-deployed configuration and does not contact the myocardial wall 10 (“BloodIn”).

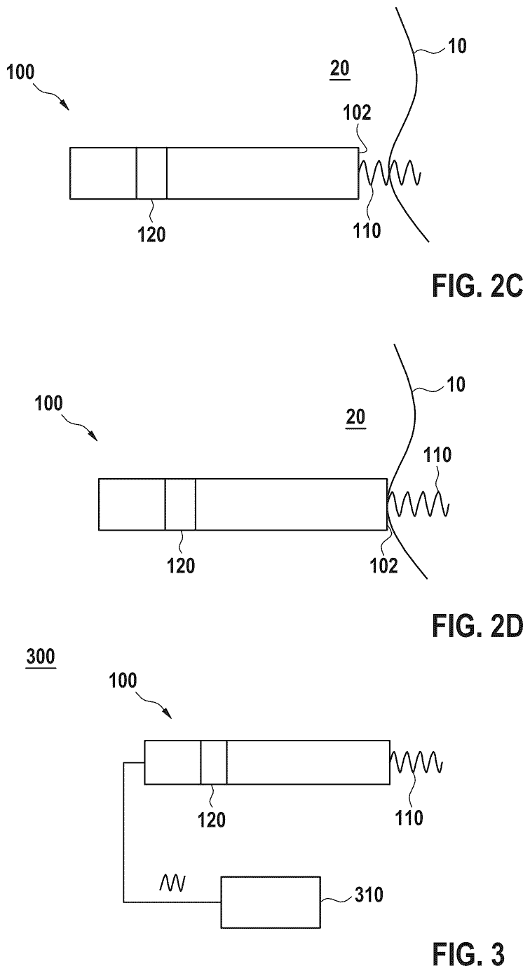

FIGS. 2A to 2D show further situations which may occur during implantation of the electrode-equipped pacing lead 100.

In FIG. 2A, the at least one first electrode 110 is in a fully deployed configuration but does not contact the myocardial wall 10 (“BloodOut”).

In FIG. 2B, the at least one first electrode 110 is in a non-deployed configuration and the pacing lead 100 (e.g., a housing thereof and/or the distal end 102 of the pacing lead 100) contacts the myocardial wall 10 (“TouchIn”).

In FIG. 2C, the at least one first electrode 110 is in a (e.g., fully) deployed configuration and is partially, for example, half, inserted into the myocardial wall 10 (“Half”).

In FIG. 2D, the at least one first electrode 110 is in a (e.g., fully) deployed configuration and is fully inserted into the myocardial wall 10 (“Full”).

The three configurations “BloodOut”, “Half” and “Full” are sometimes the cases most likely to occur in practice when fixing the electrode.

FIG. 3 shows a system 300 for characterization of an organic medium surrounding an electrode by analyzing changes of a complex impedance signal (amplitude and/or phase) according to embodiments of the present disclosure.

The system 300 includes two or more electrodes and an analysis unit 310. The two or more electrodes can be provided by the pacing lead 100 described with respect to FIGS. 1 and 2A to 2D.

In particular, the two or more electrodes may include the at least one first electrode 110 and the at least one second electrode 120.

In some embodiments, the analysis unit 310 may be included in a medical device remote from the two or more electrodes, such as a cardiac pacemaker. In further embodiments, the analysis unit 310 may be provided as a separate entity.

The analysis unit 310 is connectable to the two or more electrodes, e.g., by a two-electrode connector configured for secure attachment to the analysis unit 310 and/or the cardiac pacemaker.

The analysis unit 310 is configured to apply a measurement signal to the two or more electrodes, wherein the analysis unit 310 is further configured to determine an impedance based on the measurement signal and characterize the organic medium surrounding the at least one first electrode 110 based on an amplitude and/or phase of the impedance. For example, by analyzing the impedance, the analysis unit 310 may be configured to distinguish between the cases “BloodIn”, “BloodOut”, “TouchIn”, “Half”, and “Full” described above with respect to FIGS. 1 and 2A to 2D.

In the following, three exemplary embodiments for analyzing changes of a complex impedance signal (amplitude and/or phase) to characterize an organic medium are described. These exemplary embodiments are based on the realization that the electrical conductivity and permittivity of blood and heart muscle differ greatly at certain frequencies.

Briefly summarized, in the first embodiment, the penetration depth of electrodes in tissue is assessed in a defined frequency range (e.g., from 1 kHz (or 50 Hz) to 1 MHZ (or 10 MHz)), using at least one of a morphology, such as a number of local minima, of the phase and a maximum value of the amplitude of the impedance.

In the second embodiment, the penetration depth of electrodes in tissue is assessed at at least one certain frequency (e.g., between 1 kHz (or 50 Hz) and 1 MHz (or 10 MHz), preferably in the range of the relaxation frequency of the cardiac muscle) using the amplitude and the phase angle of the impedance.

In the third embodiment, the penetration depth of electrodes in tissue is assessed at at least one certain frequency (e.g., between 1 kHz (or 50 Hz) and 1 MHz (or 10 MHz), preferably in the range of the relaxation frequency of the cardiac muscle) using the imaginary part of the impedance.

As already mentioned above, the embodiments of the present disclosure are based on the changes of the complex impedance between the at least one first electrode (tip) and the at least one second electrode (ring) at different frequencies. The complex impedance Z is generally defined as follows:

$$\underline{Z} = Z {e}^{i φ} = Z ( cos ( φ ) + i sin ( φ ) )$$

where Z represents the amplitude of the complex impedance, while φ gives the phase. i is the imaginary unit.

The analysis of the complex impedance is based on a simplified equivalent circuit of an active lead whose head is located in the ventricle or atrium as it is shown in FIGS. 2A to 2D.

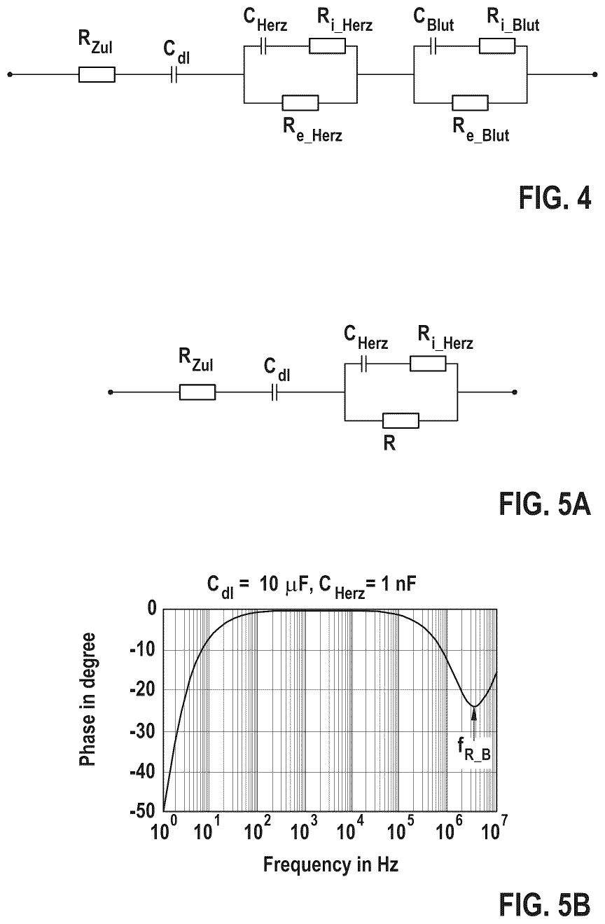

FIG. 4 shows an equivalent circuit of a lead whose distal electrode is partially or fully inserted into the myocardial wall (see FIGS. 2C and 2D). The equivalent circuit is a series circuit including (or consisting of) the lead resistance, the double layer capacitance at the surface of the screw electrode, a Fricke-Morse model (RC element) for the heart muscle and another Fricke-Morse model for the blood. The Fricke-Morse model is a simple equivalent circuit for modeling biological cells and tissues.

In FIG. 4, R_{Zul} denotes the lead resistance, Cai denotes the double layer capacitance at the surface of the screw electrode, C_{Herz} denotes the cell membrane capacitance of the heart muscle, C_{Blut} denotes the cell membrane capacitance of the blood, Ri denotes the electrical resistance of the intracellular space, and Re denotes the electrical resistance of the extracellular space. Since the resistance connected in parallel to Ca is very large, it has been omitted in the equivalent circuit.

### First Embodiment

In the first embodiment, the penetration depth of electrodes in tissue is assessed in a predetermined frequency range, using at least one of a phase morphology, such as a number of local minima, and a maximum value of the amplitude of the impedance.

Preferably, the predetermined frequency range can be from 50 Hz to 10 MHz, preferably 1 kHz to 1 MHz. Additionally, or alternatively, the predetermined frequency range can have a width of 300 kHz or more, 500 KHz or more, 800 kHz or more, or 1 MHz or more. For example, the analysis unit can be configured to sample and/or measure and/or determine the impedance over the entire predetermined frequency range for further analysis.

In one example of the first embodiment, a phase morphology of the impedance is analyzed. In particular, only the maximum and/or minimum value of the phase per measuring cycle can be evaluated.

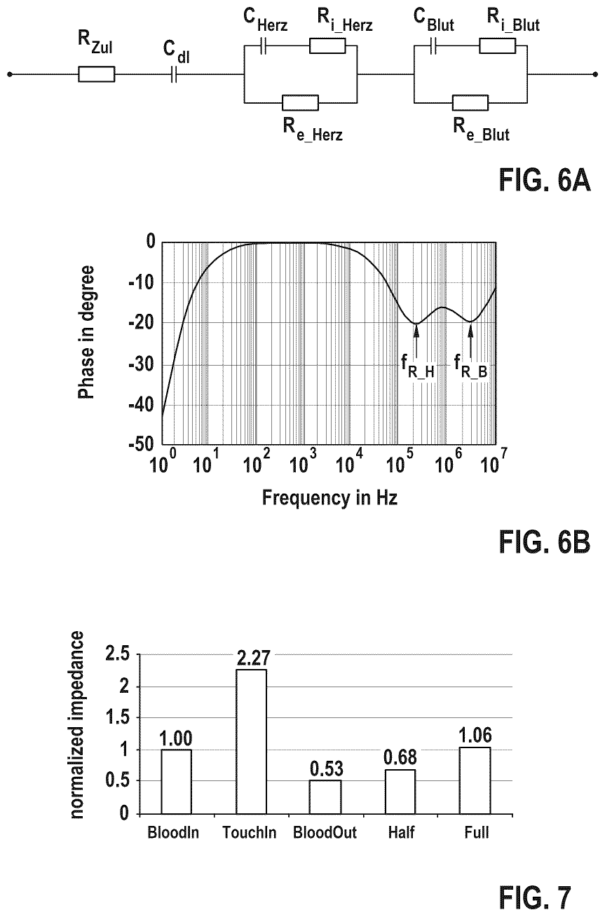

FIG. 5A shows a simplified equivalent circuit of a lead whose active electrode is located in the blood, and FIG. 5B shows a phase over a frequency range based on the equivalent circuit of FIG. 5A. FIG. 6A shows an equivalent circuit of a lead whose active electrode is located in the tissue, and FIG. 6B shows a phase over a frequency range based on the equivalent circuit of FIG. 6A.

If the fixation/position of the screw electrode is completely/only in the blood, one can expect two local minima of the phase curve of the impedance in a wide frequency range according to the equivalent circuit diagram in FIG. 5A: a local minimum due to the double layer capacitance at the surface of the screw electrode and another local minimum due to the cell membrane capacitance of the blood (FIG. 5B: only one minimum is shown at the blood relaxation frequency f_{R_B}).

On the other hand, if the screw electrode is in the tissue (e.g., myocardium), one can expect an additional local minimum of the phase curve of the impedance according to the equivalent circuit diagram in FIG. 6A. The additional local minimum is in the same frequency range at a relaxation frequency of the heart muscle f_{R_H} due to the cell membrane capacitance of the myocardium (FIG. 6B). Since the number of local minima of the phase curve depends on the predetermined frequency range, it can be generally said that if n denotes the number of local minima of the phase curve of the impedance in a defined frequency range for the case “fixation in blood”, then the number of local minima of the phase curve for the case “fixation in myocardium” is greater than n.

Consequently, at at least one frequency point, e.g., at 275 kHz, the phase when the first electrode is fixed (at least partially) in the tissue (e.g., myocardium) differs significantly, e.g., at least 5 or 10 degrees, from the phase when the first electrode is fixed/positioned (completely) in the blood.

In other words, already by means of a single measurement of the impedance it can be determined whether the first electrode is in the tissue (whether completely or partially) or not. Of course, the selected frequency point must be characteristic for the phase at which the first electrode is in the tissue, which is the case for at least one frequency point in the frequency range between 100 kHz and 400 KHz.

In another example of the first embodiment, a maximum value of the amplitude of the impedance is analyzed. In particular, only the maximum value of the amplitude per measuring cycle can be evaluated.

FIG. 7 shows maximum values of the amplitude for the cases “BloodIn”, “TouchIn”, “BloodOut”, “Half”, and “Full”. In particular, FIG. 7 is a simulation which was performed in the right ventricle of a 3D human body model. All values are normalized to the value of the case “BloodIn”. The maximum values of the amplitude were found at 500 Hz.

Due to the lower electrical conductivity of the myocardium compared to blood, the case “fixation in the myocardium” would have a higher impedance amplitude in a wide frequency range than the case where the screw electrode is located in the blood. Therefore, by analyzing the maximum value of the amplitude within the predetermined frequency range, it can be determined where (blood or tissue) and/or how deep in the tissue the screw electrode is located.

In a further example of the first embodiment, the above two examples are combined. In particular, the maximum value of the amplitude and the morphology of the phase curve can be evaluated to determined where (blood or tissue) and/or how deep in the tissue the electrode is located. For example, the penetration depth in the tissue can be assessed in the predetermined frequency range by two quantities, namely, the maximum value of the amplitude curve and the morphology (number of local minima) of the phase curve. In the following, an exemplary process is described which implements three steps in the analysis of the impedance (Step 2 is optional).

Z and n respectively denote the maximum value of the amplitude and the number of local minima of the phase in the entire predetermined frequency range.

### Step 1: Perform Measurement in “BloodIn” State

$$Z = Z 1 \to Z / Z 1 = 1$$

$$n = n 1$$

These values can be reference values for each patient. All values in Steps 2 and 3 can be normalized to these values.

### Step 2: Search for Contact to the Tissue (“TouchIn”).

Contact to the tissue is established, i.e., the case “TouchIn” is achieved if:

$$a 2 \leq Z 2 / Z 1 \leq b 2 and$$

$$n 2 > n 1$$

Here Z2 is the measured maximum value of the amplitude of the impedance in Step 2, while Z1 denotes the measured maximum value of the amplitude of the impedance in Step 1. n1 and n2 are the number of local minima of the phase curve in Step 1 and Step 2, respectively.

Step 3: Fixing the Electrode after Successfully Completing Step 2

The screw electrode is completely in the tissue, i.e., the case “Full” is achieved if:

$$a 3 \leq Z 3 / Z 1 \leq b 3 and$$

$$n 3 > n 1 ,$$

where Z3 is the measured maximum value of the amplitude in Step 3 and Z1 is the measured maximum value in Step 1. a2, b2, a3 and b3 are positive integers and can be determined from in vivo measurement results.

The analysis can be visualized in such a way that it can be used to detect changes in the organic medium at one of the electrodes, preferably the at least one first electrode.

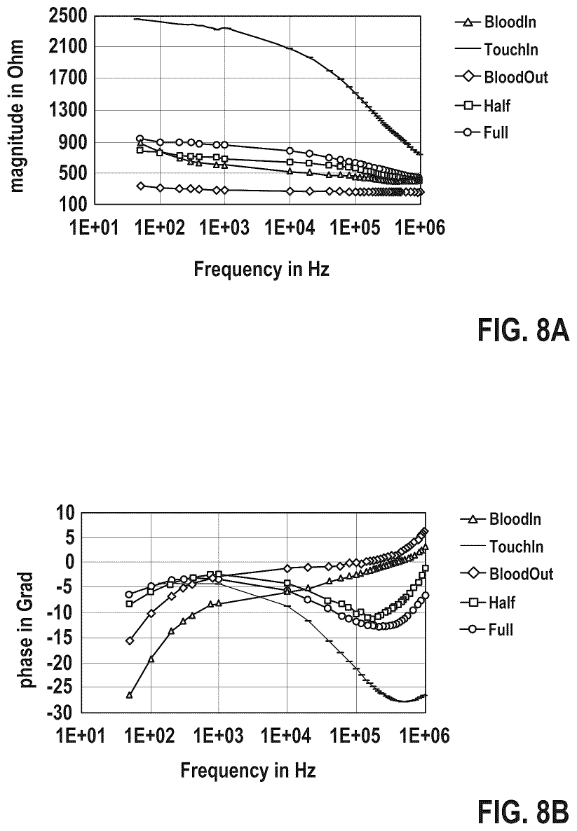

FIG. 8A shows an amplitude of the impedance over a frequency range obtained in a real experiment using the above-described first embodiment of the present disclosure. FIG. 8B shows the corresponding phase of the impedance.

FIG. 8A shows that the amplitude of “Full” is larger than the amplitudes of “Half” and “BloodOut” in the entire spectrum, confirming that the inventive concept works. Furthermore, FIG. 8B shows that the phase curves of “Half” and “Full” show relaxation in the frequency range up to 1 MHz, while “BloodOut” shows no relaxation in the same frequency range, further confirming that the inventive concept works.

### Second Embodiment

In the second embodiment, the penetration depth of electrodes in tissue is assessed at at least one certain frequency (e.g., between 1 kHz (or 50 Hz) and 1 MHz (or 10 MHz), preferably in the range of the relaxation frequency of the cardiac muscle) using the amplitude and the phase angle of the impedance.

In some implementations of the second embodiment, an optimal frequency or reference frequency can be determined in a broad frequency spectrum. The optimal frequency is a frequency at which the case “Full” differs significantly from the other cases with respect to the amplitude and the phase angle of the impedance. In some examples, this optimal frequency can be the relaxation frequency of the heart muscle, provided that the relaxation frequencies of the heart muscle and blood are sufficiently different . . . .

The relaxation frequency is defined as the frequency at which the imaginary part and approximately also the phase of the impedance have their minimum. For a simple parallel RC element, the relaxation frequency is equal to the reciprocal of the time constant τ (τ=RC).

In vivo impedance measurements of various tissues from pigs provide exemplary parameters to characterize the ischemic tissues. These parameters included relaxation frequencies of the myocardium and blood, which have the following values: 144.2±60.2 kHz (mean±standard deviation from 8 pigs) for the myocardium and 2020±420 kHz (mean±standard deviation from 4 pigs) for the blood (Casas, Oscar et al. 1999. In Vivo and In Situ Ischemic Tissue Characterization Using Electrical Impedance Spectroscopya. Annals of the New York Academy of Sciences. 873. 51-58. 10.1111/j.1749-6632.1999.tb09448.x). These two values are significantly far apart and thus fulfill the previously mentioned requirement for the application of the method to the relaxation frequency of the myocardium.

Accordingly, the penetration depth in the tissue can be assessed using the amplitude and the phase angle of the impedance at a frequency which is between 1 kHz (or 50 Hz) and 1 MHz (or 10 MHz) (ideally in the range of the relaxation frequency of the heart muscle). In the following, an exemplary process is described which implements three steps in the analysis of the impedance (Step 2 is optional).

In this embodiment, only one frequency is applied. In the following steps, Z and φ represent, respectively, the amplitude and the phase angle of the impedance at a frequency lying in the range from 1 kHz (or 50 Hz) to 1 MHz (or 10 MHz), for example, the relaxation frequency of the myocardium.

### Step 1: Perform Measurement in “BloodIn” State

$$Z = Z 1 \to Z / Z 1 = 1$$

$$φ = φ1 \to φ / φ1 = 1$$

The values measured in Step 1 are considered reference values and the values in Step 2 and Step 3 are normalized to these values.

Step 2: Search for Contact with the Tissue (“TouchIn”)

Step 2 successful if:

$$a 2 \leq Z / Z 1 \leq b 2 and$$

$$c 2 \leq φ2 / φ1 \leq d 2$$

Step 3: Fix Electrode after Step 2 has been Successfully Performed.

Electrode is completely in the tissue, i.e., case “Full” is achieved if:

$$a 3 \leq Z 3 / Z 1 \leq b 3 and$$

$$c 3 \leq φ3 / φ1 \leq d 3$$

a2, b2, c2, d2, a3, b3, c3, and d3 are positive integers and can be determined from in vivo measurement results.

The analysis can be visualized in such a way that it can be used to detect changes in the organic medium at one of the electrodes, preferably the at least one first electrode.

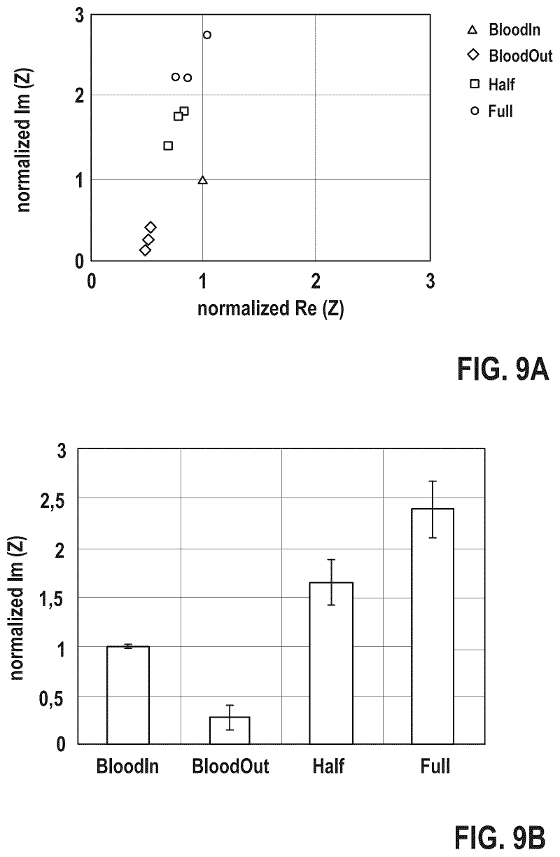

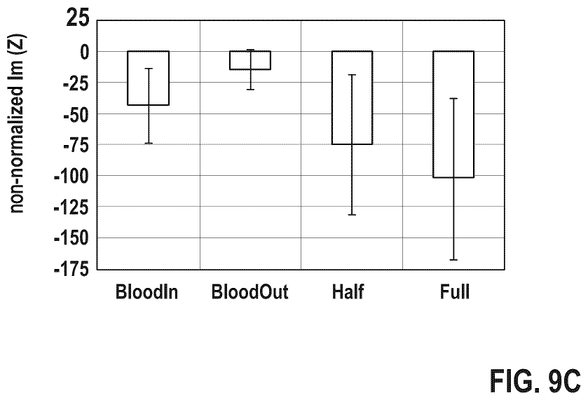

FIG. 9A shows that, for the in-vivo measurements carried out so far, the values of the imaginary part of the impedance of “Full”, “Half” and “BloodOut” at 300 kHz (relaxation frequency of the cardiac muscle obtained from the phase curve in FIG. 8B) are clearly separable from each other, further confirming that the inventive concept works.

### Third Embodiment

In the third embodiment, the penetration depth of electrodes in tissue is assessed at at least one certain frequency (e.g., between 1 kHz (or 50 Hz) and 1 MHz (or 10 MHz), preferably in the range of the relaxation frequency of the cardiac muscle) using the imaginary part of the impedance.

The third embodiment is similar the second embodiment, wherein only the imaginary part of the impedance is used in the analysis. The imaginary part of the impedance is obtained by multiplying the amplitude of the impedance by the sine of the phase angle of the impedance. And sin(x) increases with increasing x (x between 0° and) 90° and decreases with decreasing x (x between 0° and −90°).

In the range of the relaxation frequency of the myocardium, the absolute value of the phase as well as the amplitude of the impedance of “Full” are larger than the values of “Half” and

“BloodOut”. Therefore, the imaginary part of the impedance provides an increased difference between “Full” and the other two configurations “Half” and “BloodOut”.

As mentioned above, the impedance (in particular the imaginary part) needs to be measured at or near the frequency point (e.g., 275 kHz) characteristic of the phase when the first electrode is (partially or completely) in the tissue.

In the following, an exemplary process is described which implements three steps in the analysis of the impedance (Step 2 is optional).

Im denotes the imaginary part of the impedance at a frequency ranging from 1 kHz (or 50 Hz) to 1 MHz (10 MHz), for example, the relaxation frequency of the myocardium.

### Step 1: Perform Measurement in “BloodIn” State

$$Im = Im 1 \to Im / Im 1 = 1$$

The value measured in this step is considered the reference value and the values in Steps 2 and 3 are normalized to Im1.

### Step 2: Search for Contact to the Tissue (“TouchIn”)

Contact to tissue established if:

$$a 2 \leq Im 2 / Im 1 \leq b 2$$

Here Im2 is the imaginary part of the impedance measured in this step.

Step 3: Fixation of the Electrode after Successful Completion of Step 2.

The complete fixation of the screw in the tissue is successful if:

$$a 3 \leq Im 3 / Im 1 \leq b 3$$

a2, b2, a3 and b3 are positive integers and can be determined from in vivo measurement results. Here, it is important that the interval a3 to b3 of “Full” does not overlap with the intervals of “Half” and “BloodOut”. By normalizing to “BloodIn”, the probability that the intervals overlap is lower than for non-normalized values, as it is shown by the real experiment data in FIGS. 9A, 9B, and 9C (FIGS. 9B and 9C show the mean values and the standard deviation).

The analysis can be visualized in such a way that it can be used to detect changes in the organic medium at one of the electrodes, preferably the at least one first electrode.

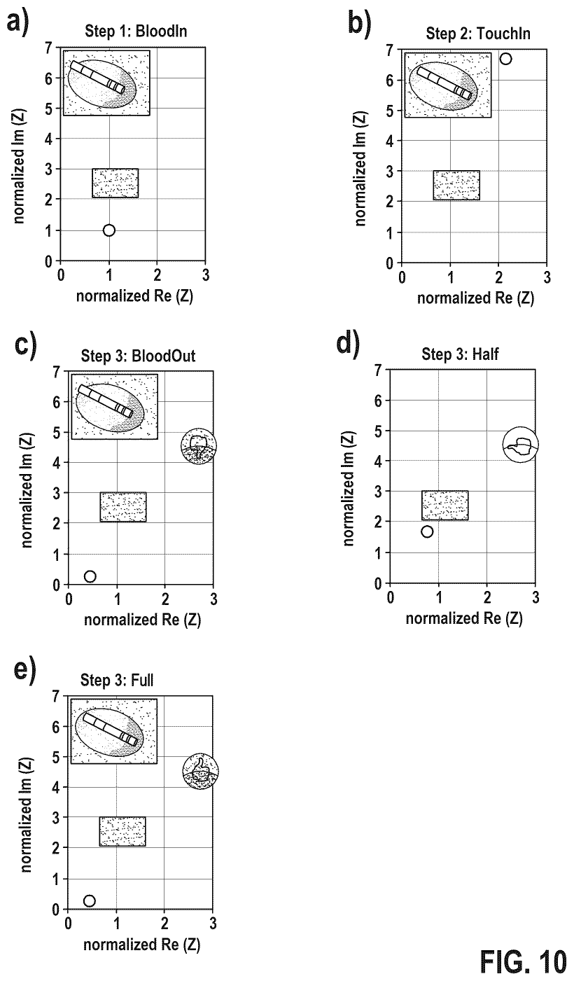

FIG. 10 shows an example for the above steps of the third embodiment:
Step 1
   (a): BloodIn
Step 2
   (b): TouchIn
Step 3
   (c): BloodOut
   (d): Half
   (e): Full

Although specific embodiments have been described to explain the inventive concept, other approaches are possible. For example, a difference between the diastolic and systolic measured values of the impedance can be evaluated. Furthermore, the measurement signal used to obtain the impedance can be a temporal signal, for example, the pacemaker pulse. In this case the impedance (defined as the peak value of the voltage signal divided by the peak value of the current signal) between the electrodes can be evaluated. Alternatively, or additionally, the morphology of the current signal can be evaluated. Finally, the impedance and the morphology of the current signal can be evaluated and/or the difference between the diastolic and systolic measured values of the impedance can be evaluated.

While the foregoing is directed to embodiments of the disclosure, other and further embodiments of the disclosure may be devised without departing from the basic scope thereof, and the scope thereof is determined by the claims that follow.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A system for characterization of an organic medium surrounding an electrode, comprising:
two or more electrodes, wherein at least one electrode of the two or more electrodes is configured to be insertable in tissue; and
an analysis unit connectable to the two or more electrodes and configured to apply a measurement signal to the two or more electrodes, wherein the analysis unit is further configured to determine an impedance between the two or more electrodes based on the measurement signal and characterize the organic medium surrounding the at least one electrode based on an amplitude and/or phase of the impedance;
and/or wherein the analysis unit is configured to apply a current pulse to at least one electrode and wherein the analysis unit is further configured to characterize the organic medium surrounding the at least one electrode based on the morphology of the applied current pulse.

2. The system of claim 1, wherein the two or more electrodes include at least one first electrode configured to be inserted in tissue and at least one second electrode spaced apart from the first electrode.

3. The system of claim 2, wherein the at least one first electrode is selected from the group consisting of a screw electrode, a tip electrode, and a hook electrode.

4. The system of claim 2, wherein the at least one second electrode is selected from the group consisting of a ring electrode, a housing, and an external electrode attachable to a body.

5. The system of claim 1, wherein the analysis unit is configured to measure and/or determine the impedance over a predetermined frequency range, in particular wherein the predetermined frequency range is from 50 Hz to 10 MHz.

6. The system of claim 5, wherein the analysis unit is configured to determine a number of local minima based on the phase over the predetermined frequency range, and to determine an organic medium surrounding the at least one electrode based on the number of local minima.

7. The system of claim 6, wherein the analysis unit is configured to determine that the at least one electrode is located in a first organic medium if the number of local minima is n, and to determine that the at least one electrode is at least partially located in a second organic medium different from the first organic medium if the number of local minima is m, wherein m≠n, and m>n particularly wherein the first organic medium is blood and the second organic medium is tissue.

8. The system of claim 5, wherein the analysis unit is configured to determine a maximum value of the amplitude of the impedance in the predetermined frequency range, and to determine an organic medium surrounding the at least one electrode based on the maximum value.

9. The system of claim 1, wherein the analysis unit (310) is configured to measure and/or determine the impedance at a reference frequency.

10. The system of claim 9, wherein the analysis unit is configured to determine an organic medium surrounding the at least one electrode based on the amplitude and/or phase of the impedance at the reference frequency.

11. The system of claim 9, wherein the analysis unit is configured to determine an organic medium surrounding the at least one electrode based on an imaginary part and/or real part of the impedance at the reference frequency.

12. The system of claim 9, wherein the reference frequency corresponds to a relaxation frequency of a cardiac muscle, such as a myocardial wall.

13. The system of claim 1, comprising:
a pacing lead carrying the two or more electrodes; and
a pacemaker connected to the pacing lead, in particular wherein the analysis unit is included in the pacemaker.

14. A method for characterization of an organic medium surrounding an electrode, comprising:
applying a measurement signal to two or more electrodes, wherein at least one electrode of the two or more electrodes is configured to be insertable in tissue; and
determining an impedance between the two or more electrodes based on the measurement signal; and
characterizing the organic medium surrounding the at least one electrode based on an amplitude and/or phase of the impedance.

15. A machine-readable medium, comprising instructions executable by one or more processors to implement the method for characterization of an organic medium surrounding an electrode of claim 14.
